(19) United States
(12) Patent Application Publication
(10) Pub. No.: US 2009/0233973 A1
(43) Pub. Date: Sep. 17, 2009

(54) EPOTHILONE DERIVATIVES FOR THE TREATMENT OF MULTIPLE MYELOMA

(76) Inventors: Boris Lin, Indianapolis, IN (US); Kenneth C. Anderson, Wellesley, MA (US); James Douglas Griffin, Brookline, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/474,555

(22) Filed: May 29, 2009

Related U.S. Application Data
(63) Continuation of application No. 10/530,857, filed on Aug. 16, 2005.
(60) Provisional application No. 60/417,916, filed on Oct. 11, 2002.

(51) Int. Cl. A61K 31/427; A61P 35/00

(52) U.S. Cl. 514/365

(57) ABSTRACT

The present invention relates to a method of treating a warm-blooded animal, especially a human, having myeloma, especially myeloma which is resistant to conventional cytotoxic chemotherapy, comprising administering to said animal a therapeutically effective amount of an epothilone, especially an epothilone of formula I

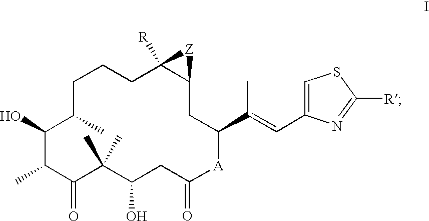

to a combination comprising an epothilone, for simultaneous, separate or sequential use; and to a pharmaceutical composition and a commercial package comprising said combination.

This is a continuation of application Ser. No. 10/530,857 filed on Aug. 16, 2005, which is a National Stage of International Application No. PCT/IB03/04480 filed on Oct. 10, 2003, which claims benefit of U.S. Provisional Application No. 60/417,916 filed Oct. 11, 2002, which in their entirety are herein incorporated by reference.

The present invention relates to a method of treating a warm-blooded animal, especially a human, having myeloma, especially myeloma which is resistant to conventional cytotoxic chemotherapy, comprising administering to said animal a therapeutically effective amount of an epothilone, especially an epothilone of formula I as defined herein; to a combination comprising an epothilone and a compound selected from the group consisting of alkylating agents, corticosteroids and anthracyclines, and optionally at least one pharmaceutically acceptable carrier, for simultaneous, separate or sequential use; and to a pharmaceutical composition and a commercial package comprising said combination.

The taxanes, such as paclitaxel and docetaxel, represent a class of microtubule stabilizing agents that is commonly used in a number of proliferative diseases, e.g., solid tumor diseases like ovarian cancer. However, the taxanes have not shown great promise in the treatment of myeloma. The epothilones, e.g., epothilones A, B and D, but also analogues thereof, represent a new class of microtubule stabilizing agents (see Gerth, K. et al., J. Antibiot. 49, 560-3 (1996); or Hoefle et al., DE 41 38 042). Surprisingly, it was now found that epothilones, especially the epothilones of formula I as defined herein and, in particular, epothilone B, directly inhibit the growth and survival of myeloma cells.

Furthermore, adherence of patient multiple myeloma cells to bone marrow stromal cells (BMSCs), enhances the ability of epothilones to inhibit multiple myeloma cell proliferation and to promote cell death proliferation of myeloma cells that are adherent to BMSCs.

Hence, the invention relates to a method of treating myeloma, especially myeloma which is resistant to conventional cytotoxic chemotherapy, comprising administering a therapeutically effective amount of an epothilone, preferably a therapeutically effective amount of an epothilone of formula I

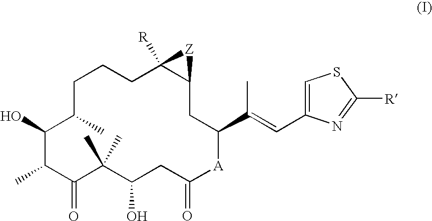

wherein A represents O or NR_{N}, wherein R_{N} is hydrogen or lower alkyl, R is hydrogen or lower alkyl, R′ is methyl, methoxy, ethoxy, amino, methylamino, dimethylamino or methylthio, and Z is O or a bond,
or a pharmaceutically acceptable salt thereof to a warm-blooded animal, preferably a human, in need thereof.

The present invention pertains in particular to a method of treating myeloma wherein

(a) overexpression of the multi-drug resistance protein p170 is observed, and/or
(b) the myeloma is resistant to a taxane, e.g., paclitaxel or docetaxel.

The term “myeloma” as used herein relates to a tumor composed of cells of the type normally found in the bone marrow. The term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia. Multiple myeloma is incurable by the use of conventional cytotoxic and high dose chemotherapies.

Throughout the present specification and claims myeloma means preferably multiple myeloma (MM).

Unless stated otherwise, in the present disclosure organic radicals and compounds designated “lower” contain not more than 7, preferably not more than 4, carbon atoms.

A compound of formula I wherein A represents O, R is hydrogen, R′ is methyl and Z is O is known as epothilone A; a compound of formula I wherein A represents O, R is methyl, R′ is methyl and Z is O is known as epothilone B; a compound of formula I wherein A represents O, R is hydrogen, R′ is methyl and Z is a bond is known as epothilone C; a compound of formula I wherein A represents O, R is methyl, R′ is methyl and Z is a bond is known as epothilone D.

Epothilone derivatives of formula I wherein A represents O or NR_{N}, wherein R_{N} is hydrogen or lower alkyl, R is hydrogen or lower alkyl, R′ is methyl and Z is O or a bond, and methods for the preparation of such epothilone derivatives are in particular generically and specifically disclosed in the patents and patent applications WO 93/10121, U.S. Pat. No. 6,194,181, WO 98/25929, WO 98/08849, WO 99/43653, WO 98/22461 and WO 00/31247 in each case in particular in the compound claims and the final products of the working examples, the subject-matter of the final products, the pharmaceutical preparations and the claims is hereby incorporated into the present application by reference to this publications. Comprised are likewise the corresponding stereoisomers as well as the corresponding crystal modifications, e.g. solvates and polymorphs, which are disclosed therein. Epothilone derivatives of formula I, especially epothilone B, can be administered as part of pharmaceutical compositions which are disclosed in WO 99/39694.

Epothilone derivatives of formula I wherein A represents O or NR_{N}, wherein R_{N} is hydrogen or lower alkyl, R is hydrogen or lower alkyl, R′ is methoxy, ethoxy, amino, methylamino, dimethylamino or methylthio, and Z is O or a bond, and methods for the preparation and administration of such epothilone derivatives are in particular generically and specifically disclosed in the patent application WO99/67252, which is hereby incorporated by reference into the present application. Comprised are likewise the corresponding stereoisomers as well as the corresponding crystal modifications, e.g. solvates and polymorphs, which are disclosed therein.

The transformation of epothilone B to the corresponding lactam is disclosed in Scheme 21 (page 31, 32) and Example 3 of WO 99/02514 (pages 48-50). The transformation of a compound of formula I which is different from epothilone B into the corresponding lactam can be accomplished analogously. Corresponding epothilone derivatives of formula I wherein R_{N} is lower alkyl can be prepared by methods known in the art such as a reductive alkylation reaction starting from the epothilone derivative wherein R_{N} is hydrogen.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

In one preferred embodiment of the invention, an epothilone derivative of formula I is employed wherein A represents O, R is lower alkyl, especially methyl, ethyl or n-propyl, or hydrogen, R′ is methyl and Z is O or a bond. More preferably, an epothilone derivative of formula I is employed wherein A represents O, R is methyl, R′ is methyl and Z is O, which compound is also known as epothilone B.

The term “treatment” as used herein comprises the treatment of patients having myeloma or being in a pre-stage of said disease which effects the delay of progression of the disease in said patients and aims preferably to effect a complete response to the treatment, a partial response to the treatment or to effect a stable disease.

The term “complete response” as used herein means in particular to the resolution of all measurable or evaluable disease.

The term “partial response” as used herein means in particular a greater than or equal to 50% reduction in measurable or evaluable disease in the absence of progression in any particular disease site.

The term “stable disease” as used herein means in particular a less than 50% decrease or less than 25% increase in measurable or evaluable disease.

The present invention pertains also to a combination comprising (a) an epothilone and (b) at least one compound selected from the group consisting of alkylating agents, corticosteroids, and anthracyclines. in particular, for the simultaneous, separate or sequential use in the treatment of myeloma.

The term “alkylating agent” as used herein includes, but is not limited to, alkyl sulfonates, aziridines, epoxides, ethylenimines, methylmelamines, nitrogen mustards, nitrosoureas, imidazotetrazinones, dacarbazine, mannomustine, mitobronitol, mitolactol, pipobroman and procarbazine.

The term “alkyl sulfonates” as used herein includes, but is not limited to, busulfan, improsulfan and piposulfan.

The term “aziridines” as used herein includes, but is not limited to, benzodepa, carboquone, meturedepa and uredepa.

The term “ethylenimines and methylmelamines” as used herein includes, but is not limited to, altretamine, triethylenemelamine, triethylenephosphoramide, triethylenethiophosphoramide and trimethylolmelamine.

The term “nitrogen mustards” as used herein includes, but is not limited to, chlorambucil, chlomaphazine, cyclophosphamide, estramustine, ifosfamide, mechlorethamine, mechlorethamine oxide hydrochloride, melphalan, novembichin, phenesterine, prednimustine, trofosfamide and uracil mustard.

The term “nitrosoureas” as used herein includes, but is not limited to, carmustine, chlorozotocin, cytemustine, fotemustine, lomustine (CCNU), nimustine and ranimustine.

The term “imidazotetrazinones” as used herein includes, but is not limited to, temozolomide and mitozolomide.

“Temozolomide” is described in U.S. Pat. No. 5,260,291. The synthesis of temozolomide is well known e.g., Wang et al., J. Org. Chem. 1997, 62, 7288-7294). Temozolomide is commercially available e.g. under the trademark of TEMODAL™, TEMODAR™, or TEMOXOL™ and can be administered, e.g., as described in U.S. Pat. No. 5,942,247 or according to the package insert information. The term “lomustine” means a compound as described and prepared e.g. in Johnson P et al., J. Med. Chem. 1966, 9, 892. Lomustine is commercially available under the trademark BETULUSTINE™ and can be administered according to the package insert information.

The term “anthracyclines” as used herein includes, but is not limited to, doxorubicine and daunorubicine.

Furthermore, the structure of the active agents mentioned herein by name may be taken from the actual edition of the standard compendium “The Merck Index” or from databases, e.g. Patents International (e.g. IMS World Publications). The corresponding content thereof is hereby incorporated by reference. Any person skilled in the art is fully enabled, based on these references, to manufacture and test the pharmaceutical indications and properties in standard test models, both in vitro and in vivo.

A combination comprising (a) an epothilone and (b) at least one compound selected from the group consisting of alkylating agents, corticosteroids and anthracyclines, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATION OF THE INVENTION can be a combined preparation or a pharmaceutical composition.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a non-effective dosage of one or both of the first and second active ingredient, and especially a strong synergism the first and second active ingredient.

Additionally, the present invention provides a method of treating myeloma comprising administering a COMBINATION OF THE INVENTION in an amount which is jointly therapeutically effective against myeloma to a warm-blooded animal in need thereof.

The person skilled in the pertinent art is fully enabled to select relevant test models to prove the hereinbefore and hereinafter mentioned beneficial effects on myeloma of an epothilone or of a COMBINATION OF THE INVENTION. The pharmacological activity of an epothilone or a COMBINATION OF THE INVENTION may, for example, be demonstrated in a suitable clinical study or by means of the Examples described below. By the methods described below it can be shown, e.g., that epothilone B inhibits the growth and survival of MM cells with an IC90 between 1 and 10 nM. Epothilon B induces G2M arrest in MM cells with subsequent apoptosis. Suitable clinical studies are, for example, open label non-randomized, dose escalation studies in patients with advanced myeloma. Such studies prove in particular the synergism observed with the COMBINATIONS OF THE INVENTION. The beneficial effects on myeloma can be determined directly through the results of such studies or by changes in the study design which are known as such to a person skilled in the art. For example, one combination partner can be administered with a fixed dose and the dose of a second combination partner is escalated until the Maximum Tolerated Dosage (MTD) is reached. Alternatively, a placebo-controlled, double blind study can be conducted in order to prove the benefits of the COMBINATION OF THE INVENTION mentioned herein.

It is one objective of this invention to provide a pharmaceutical composition comprising a quantity, which is jointly therapeutically effective against myeloma comprising the COMBINATION OF THE INVENTION. In this composition, the combination partners can be administered together, one after the other or separately in one combined unit dosage form or in two separate unit dosage forms. The unit dosage form may also be a fixed combination.

The pharmaceutical compositions for separate administration of the combination partners and for the administration in a fixed combination, i.e. a single galenical composition comprising at least two combination partners, according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of treatment of myeloma according to the present invention may comprise (i) administration of a combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of the epothilones and of the combination partners employed in the COMBINATION OF THE INVENTION may vary depending on the particular compound or pharmaceutical composition employed, the mode of administration, the type of the myeloma being treated and the severity of the myeloma being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of an epothilone or of the single active ingredients of the COMBINATION OF THE INVENTION required to prevent, counter or arrest the progress of the condition. Optimal precision in achieving concentration of the active ingredients within the range that yields efficacy without toxicity requires a regimen based on the kinetics of the active ingredients' availability to target sites.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

If the warm-blooded animal is a human, the dosage of a compound of formula I is preferably in the range of about 0.1 to 75, preferably 0.25 to 50, e.g. 2.5 or 6, mg/m² once weekly for two to four, e.g. three, weeks, followed by 6 to 8 days off in the case of an adult patient.

In one embodiment of the invention, epothilone B is administered weekly in a dose that is between about 0.1 to 6 mg/m², preferably between 0.1 and 3 mg/m², e.g. 2.5 mg/m², for three weeks after an interval of one to six weeks, especially an interval of one week, after the preceding treatment. In another embodiment of the invention said epothilone B is preferably administered to a human every 18 to 24 days in a dose that is between about 0.5 and 7.5 mg/m².

Temozolomide is preferably administered daily at a dose of 50 to 300 mg/m²/day, most preferably 200 mg/m²/day in cycles of 5 consecutive days per 28 day cycle. For patients who had prior chemotherapy, treatment is generally started at 150 mg/m²/day.

Lomustine is preferably administered at a single dose of 60 to 180 mg/m² once every six weeks preferably at a dose of 130 mg/m².

Moreover, the present invention provides a commercial package comprising as active ingredients the COMBINATION OF THE INVENTION, together with instructions for simultaneous, separate or sequential use thereof in the treatment of myeloma.

The present invention also provides the use of a compound of formula I as defined herein and the use of a COMBINATION OF THE INVENTION for the preparation of a medicament for the treatment of myeloma.

### EXAMPLES

### General

RPMI 8226 and U266 human MM cell lines can be obtained from the American Type Culture Collection (ATCC) of Rockville, Md. Patient derived MM cells are purified from patient BM samples, as described by Y. T. Tai, G. Teoh, Y. Shima, et al in J. Immunol. Methods 235:11, 2000. All human MM cell lines are cultured in RPMI-1640 media (Sigma Chemical, St. Louis, Mo.), containing 10% fetal bovine serum (FBS), 2 mmol/L L-glutamine (L-glut, GIBCO, Grand Island, N.Y.), 100 U/mL penicillin and 100 mg/mL streptomycin (P/S, GIBCO). MM patient cells are >95% CD38+, CD45RA−. Bone marrow stromal cells (BMSCs) are prepared from aspirates of MM patients as well as healthy donors as described by D. Gupta, S. Treon, Y. Shima, et al in Leukemia, 2001 and S. Gartner and H. S. Kaplan in Proc. Natl. Acad. Sci. USA 77:4756, 1980. Cells are cultured in ISCOVE's modified Dulbecco media containing 20% FBS, 2 mmol/L L-glut, and 100 ug/mL P/S. Human umbilical vein endothelial cells (HUVEC P168) are purchased from Clonetics, Biowhittaker, and maintained in EGM-2MV media (Clonetics, Biowhittaker). The epothilones are dissolved in dimethyl sulfoxide (DMSO Sigma) and stored as a stock solution at −20° C. until used. For all assays, the compound is diluted in culture medium to concentrations ranging, e.g., from 0.01 to 100 μM.

Cytokine levels are measured in supernatants from the co-culture system described above. VEGF and IL-6 concentrations are measured using commercially available ELISA kits (R&D Systems).

### Cell Protein Lysates, Immunoprecipitation and Western Blot Analysis

MM cells are starved for 12 h in RPMI with 10% FBS, and then incubated for 1 h in RPMI-1640 without FBS in the presence of an epothilone or DMSO control. These cells are subsequently stimulated with 100 nM VEGF₁₆₅ as described by K. Podar, Y. T. Tai, et al in Blood 98:428, 2001. Cells are then lysed in RIPA buffer containing 1 mM PMSF, 1 mM Sodium vanadate, and a protease inhibitor cocktail (Boehringer Mannheim). Lysates are either analyzed directly on a sodium dodecyl sulfate-polyacrylamide gel (SDS-PAGE gel) or incubated overnight with an antibody (Ab) against Flt-1, as well as protein G plus-Agarose (both from Santa Cruz Biotechnology, CA). Whole cell lysates (30 μg per lane) or immunoprecipitates are analyzed on an 8 to 10% SDS-PAGE gel; transferred onto Hybond C Super paper (Amersham, Arlington Heights, Ill.); then probed with a murine MoAb against phospho-ERK, a murine MoAb against phospho-tyrosine residues, or Abs against Flt-1 or ERK2 (Santa Cruz); and detected using an HRP-conjugate anti-murine or anti-rabbit Ab (both from Santa Cruz) and enhanced chemiluminescence (ECL) substrate solution (Amersham).

### Western Blotting

Protein lysates from drug-treated and control MM cells are prepared using RIPA buffer in the presence of a protease inhibitor cocktail (Roche), 1 mM PMSF, and 1 mM sodium orthovanadate. Lysates are either analyzed directly on sodium dodecyl sulfate-polyacrylamide (SDS-PAGE) gel; transferred onto Hybond C Super paper (Amersham, Arlington Heights, Ill.); probed with a murine MoAb against bcl-2 (Santa Cruz, Santa Cruz, Calif.), bax (Santa Cruz), or PARP (Blomol, West Grove, Pa.), or rabbit polyclonal Ab against caspase 3 (Santa Cruz), as well as goat polyclonal Ab against actin, and detected using an HRP-conjugated anti-murine or anti-goat Ab (both from Santa Cruz) and enhanced chemiluminescence (ECL) substrate solution (Amersham).

### Proliferation and Cell Viability Assays

MM cells are first starved for 12 h in RPMI-1640 media containing 10% fetal bovine serum, and then plated into 96-well microtiter plates (Costar, Cambridge, Mass.), in the presence of drug or DMSO control. Experiments are also performed in the presence or absence of VEGF₁₆₅ (R and D Systems). Proliferation is measured by the incorporation of [³H]-thymidine (NEN Products, Boston, Mass.). Specifically, cells are pulsed with [³H]-thymidine (0.5 μCi/well) for the last 6 h of 48 h cultures, harvested onto glass filters with an automatic cell harvester (Cambridge Technology, Cambridge, Mass.), and counted using a LKB Betaplate scintillation counter (Wallac, Gaithersburg, Md.). Measurement of cell viability is performed calorimetrically by MTS assay, utilizing the CellTiter96 AQᵤₑₒᵤₛ One Solution Reagent (Promega, Madison, Wis.). Cells are exposed to the MTS for the last 2 h of 48 h cultures, and absorbance is measured using an ELISA plate reader (Molecular Devices Corp., Sunnyvale, Calif.) at OD of 570 nm.

### Cell Cycle Analysis

MM cells (1×10⁶ cells) are cultured in the presence of Epothilone B or DMSO control for 24, 48 and 72 h. Cells are then washed with phosphate buffered saline (PBS), fixed with 70% ethanol, and treated with RNAse (Sigma). Cells are next stained with propidium iodide (PI, 5 μg/mL), and the cell cycle profile is determined using the M software on an Epics flow cytometer (Coulter Immunology, Hialeah, Fla.).

### Example 1

### Proliferation of MM Cells in an Adhesion System

BMSCs (1×10⁴ cells/well) are plated into 96-well microtiter plates and incubated at 37° C. for 24 h in ISCOVE's media (20% FBS). MM cells are then added to the BMSC-containing wells (5×10⁴ cells/well), in the presence of an epothilone or DMSO control. When MM.1S cells are used, both BMSCs and MM cells are starved for 12 h in RPMI-1640 media containing 2% FBS. When patient PCL cells are used, the co-cultures are performed in RPMI media containing 10% FBS. BMSCs and MM cells are also cultured separately to serve as controls. After 48 h, proliferation and cell viability are analyzed as described above. To ensure that all cells are collected for the proliferation assay, 10× Trypsin (Sigma) is added to each well 10 minutes prior to harvesting.

### Example 2

### Proliferation of MM Cells in a Modified Boyden Chamber Transwell System

Proliferation is measured in a modified Boyden chamber transwell system, using 24-well plates with a 0.4 mm pore size inserts (Costar). BMSCs (4×10⁴ cells/well) are plated in the lower chamber, starved, and incubated in an epothilone as described above. MM cells (20×10⁴ cells/ml) are then placed in the upper chamber (insert), and [³H]-thymidine uptake in the individual chambers is measured at 48 h as described above.

### Example 3

### Measurement of Cytokine Concentrations

Cytokine levels were measured in supernatants from the co-culture system described above. VEGF and IL-6 concentrations were measured using commercially available ELISA kits (R&D Systems).

### Example 4

### Effect of Epothilone B on Proliferation MM.1S Cells

MM.1S cells are placed in the upper chamber of a transwell co-culture system in order to preclude direct contact between MM cells and BMSCs, but nonetheless allow for diffusion of humoral factors. Despite the lack of contact between the two cell types, uptake of [³H]-dT by MM.1S cells incubated with BMSCs is increased by 2.2-fold (p<0.0001) at 48 h. By contrast, the BMSCs in the co-culture system do not show a significant increase in [³H]-dT uptake. It can be shown by this co-culture system that epothilone B reduces proliferation of MM.1S cells.

### Example 5

### Effect of Epothilone B on Human MM Cells In Vivo

Mice are inoculated subcutaneously into the right flank with 3×107 MM cells in 100 mL of RPMI 1640, together with 100 uL matrigel basement membrane matrix (Becton Dickinson, Bedford, Mass.). On day 6 post injection, mice are assigned into two treatment groups receiving Epothilone B, or into a control group. Treatment with Epothilone B is given intravenously once weekly via tail vein at 2.5 mg/kg for 4 weeks, starting on day +6, or as a one-time 4 mg/kg dose on day +6. The control group receive the vehicle alone (30% PEG-300 in 0.9% sodium chloride) weekly. Caliper measurements of the longest perpendicular tumor diameters are performed twice per week to estimate the tumor volume, using the following formula: 4/3×(width/2)2×(length/2), representing the three-dimensional volume of an ellipse. Animals are sacrificed when their tumor reached 2 cm or when the mice become moribund. Survival is evaluated from the first day of tumor injection until death.

What is claimed:

1. A method of treating a warm-blooded animal having myeloma which consists of administering to the warm-blooded animal a therapeutically effective dose of epothilone B or a pharmaceutically acceptable salt thereof.

2. The method according to claim 1 wherein the warm-blooded animal is a human.

3. The method according to claim 1 wherein the myeloma is resistant to conventional cytotoxic chemotherapy.

4. The method according to claim 1 wherein overexpression of the multi-drug resistance protein p170 is observed.

5. The method according to claim 1 wherein the myeloma is resistant to a taxane.

6. The method according to claim 1 wherein the disease is multiple myeloma.

7. The method according to claim 1 comprising administering epothilone B weekly in a dose that is between about 0.1 to 6 mg/m² for three weeks after an interval of one to six weeks after the preceding treatment.
